(19) United States
(12) Patent Application Publication
(10) Pub. No.: US 2005/0256541 A1
(43) Pub. Date: Nov. 17, 2005

(54) Catheter with temporary stimulation electrode

(75) Inventor: Paul H. Stypulkowski, North Oaks, MN (US)

Correspondence Address: SHUMAKER & SIEFFERT, P. A., 8425 SEASONS PARKWAY, SUITE 105, ST. PAUL, MN, 55125, US

(73) Assignee: Medtronic, Inc., Minneapolis, MN (US)

(21) Appl. No.: 11/119,390

(22) Filed: Apr. 29, 2005

Related U.S. Application Data
(60) Provisional application No. 60/566,955, filed on Apr. 30, 2004.

(51) Int. Cl. A61N 1/18

(52) U.S. Cl. 607/3; 600/547; 600/554

(57) ABSTRACT

A catheter has at least one removable, temporary stimulation electrode for positioning of the catheter prior to delivery of a therapeutic substance, such as pharmaceutical or biological therapy, to the nervous system of a patient. A stylet having at least one temporary stimulation electrode disposed at its distal end is removably positioned within a lumen of the catheter. A test stimulation is delivered via the temporary stimulation electrode to produce a patient response. If the patient response is not consistent with proper positioning of the distal end of the catheter with respect to the target tissue, the distal end of the catheter is repositioned and another test stimulation is delivered. This process is repeated until the patient response is consistent with proper positioning of the distal end of the catheter. The stylet and its associated temporary stimulation electrode are then removed from the lumen, leaving the catheter in position with respect to the target tissue. A therapeutic substance may then be delivered to the target tissue via the lumen.

This application claims the benefit of U.S. provisional application No. 60/566,955, filed Apr. 30, 2004, the entire content of which is incorporated herein by reference.

### TECHNICAL FIELD

The invention relates to neurostimulation therapy and, more particularly, to placement of a catheter for delivery of a therapeutic substance to the central nervous system.

### BACKGROUND

Delivery of therapeutic substances such as pharmaceutical or biological therapies to the brain, spinal column or other part of the central nervous system have been used to treat a variety of neurological symptoms or conditions such as chronic pain, spasticity, dystonia, Parkinson's disease, or to provide pain relief during surgery. Delivery of electrical stimulation to targets in the central nervous system has also been used to treat these and a number of other neurological conditions. Electrical stimulation therapy may be delivered via a stimulation electrode on an electrical lead located proximate to target tissue in the brain, spinal column, pelvic nerves, stomach, or other organ of a patient. Therapeutic substances, such as pharmaceutical therapies (e.g., anesthetics or narcotics) biological therapies (e.g., gene therapy agents or growth factors) or other neuropharmacologic agents (i.e., agents that effect neurotransmitter activity or metabolism) may be delivered via a catheter inserted within or near the target tissue. The electrodes or the catheter are carefully positioned to ensure that the therapy is delivered to the intended target tissue.

Target verification in the case of therapeutic substances to target tissue within the brain first involves localization or mapping of functional brain structures. Anatomical localization of brain targets can be accomplished using anatomical brain atlases, imaging by means of positive contrast x-rays, CT or MRI under stereotactic conditions. Such standard well-known imaging techniques are used to make an initial determination of location coordinates for the target tissue to which the catheter will be directed during the surgical implant procedure.

After insertion of the catheter into the area of the brain identified as described above, small quantities of a therapeutic substance may then be delivered to achieve more precise target verification. If the delivered therapy has the desired effect, target verification can be achieved. However, because therapeutic substances often require an extended period of time to take effect, this technique may not be viable as a means of target verification during the surgical procedure.

Another method of achieving more precise target verification involves electrical test stimulation of the identified brain structures. The test stimulation is delivered via a electrical stimulation lead inserted into the target tissue. When the patient response to electrical test stimulation is consistent with appropriate lead placement, target verification can be achieved. The electrical stimulation lead is then removed from the body, and a catheter is inserted for delivery of the therapeutic substance.

### SUMMARY

In general, the invention is directed to devices and methods for positioning a medical catheter within the nervous system of a patient for delivery of therapeutic substances. A catheter having a lumen extending therethrough for delivery of therapeutic substances includes a stylet removably positioned within the lumen. The stylet includes at least one temporary stimulation electrode at its distal end. In use, the distal end of the catheter is positioned proximate target tissue within the brain or other part of the central nervous system of the patient, such as the spinal column. The temporary stimulation electrode can be used to deliver a test stimulation to verify whether the catheter is properly positioned with respect to the target tissue.

If the patient response to the test stimulation is consistent with accurate target localization, it may be determined that the catheter is properly positioned with respect to the target tissue. However, if the patient response to the test stimulation is not consistent with proper positioning of the catheter, the catheter may be repositioned and the temporary stimulation electrode may deliver additional test stimulations. After target verification is achieved, the stylet may be removed from the lumen, while leaving the catheter in place for delivery of the therapeutic substance.

In one embodiment, the invention provides a device comprising a catheter having a proximal end and a distal end and a lumen extending between the proximal end and the distal end of the catheter to deliver a therapeutic substance to target tissue, a stylet removably positioned within the lumen and having a proximal end and a distal end, wherein the distal end of the stylet extends beyond the distal end of the catheter, and at least one stimulation electrode disposed at the distal end of the stylet to verify proper positioning of the distal end of the catheter with respect to the target tissue.

In another embodiment, the invention provides a method comprising introducing a catheter having a temporary stimulation electrode removably positioned therein to target tissue within a body of a patient such that a distal end of the temporary stimulation electrode extends beyond a distal end of the catheter, delivering a test stimulation via the temporary stimulation electrode to produce a patient response, determining, based on the patient response, whether the catheter is properly positioned with respect to the target tissue, and removing the temporary stimulation electrode from the catheter when the catheter is properly positioned.

In another embodiment, the invention provides a device comprising a catheter having a proximal end and a distal end and a lumen extending between the proximal end and the distal end of the catheter to deliver a therapeutic substance to target tissue, a stiffening stylet removably positioned within the lumen and having a proximal end and a distal end, wherein the distal end of the stylet extends beyond the distal end of the catheter, and at least one stimulation electrode disposed at the distal end of the stylet to verify proper positioning of the distal end of the catheter with respect to the target tissue.

The invention may provide one or more advantages. For example, introduction of a test stimulation electrode via a catheter permits quick verification of proper catheter positioning. In particular, patient response to electrical test stimulation can be evaluated almost immediately, in comparison to evaluation of patient response to therapeutic substances, which may require an extended period of time and thereby prolong the placement procedure. In addition, the invention avoids the need to separately deploy the catheter and an electrode lead. Instead, if the test electrode introduced via the catheter indicates proper positioning, the electrode is withdrawn but the catheter simply remains in place at the target site.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a perspective diagram illustrating a system including a catheter having a temporary stimulation electrode.

FIG. 2 is a diagram illustrating one embodiment of a catheter having a temporary stimulation electrode positioned within the brain of a patient.

FIG. 3 is a diagram illustrating an embodiment of a catheter connected to deliver a therapeutic substance to the brain of a patient.

FIGS. 4A-4C are cross-sectional side views illustrating different embodiments of catheters with temporary stimulation electrodes.

FIG. 5A is a cross-sectional side view of a catheter with a temporary stimulation electrode.

FIG. 5B is a cross-sectional side view of the catheter of FIG. 5A with the temporary stimulation electrode removed.

FIG. 5C is a cross-sectional side view of a stylet and temporary stimulation electrode removed from the catheter of FIG. 5A.

FIG. 6 is a flow diagram illustrating an exemplary technique for positioning a catheter using a temporary stimulation electrode and delivering a therapeutic substance to a patient via the catheter.

FIG. 7 is a flow diagram illustrating another exemplary technique for positioning a catheter using a temporary stimulation electrode and delivering a therapeutic substance to a patient via the catheter.

### DETAILED DESCRIPTION

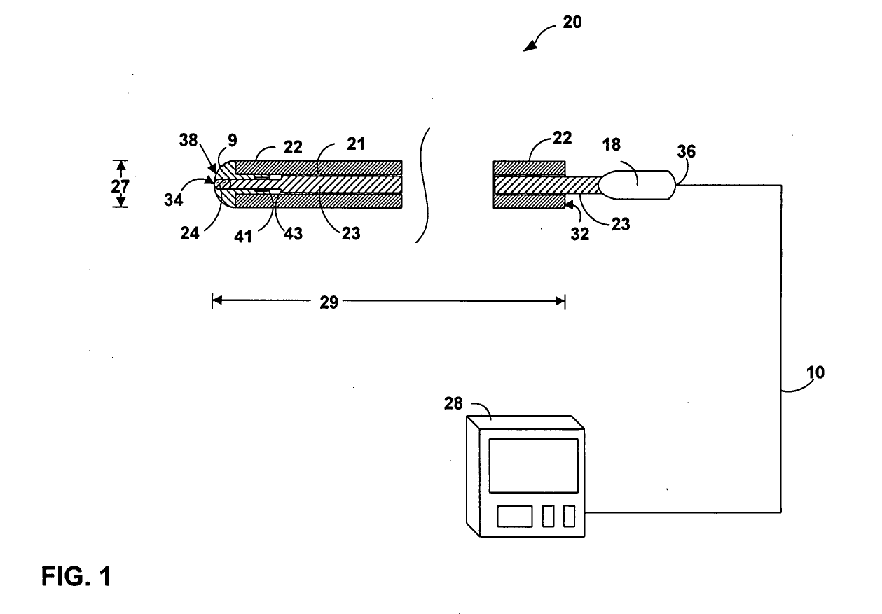

FIG. 1 is a perspective diagram illustrating an exemplary system 20 that includes a catheter 22 having a temporary stimulation electrode 24. Catheter 22 includes a lumen 21 extending from the proximal end 32 to the distal end 38 of the catheter 22. End cap 9 is disposed at the distal end 38 of catheter 22. Catheter 22 has a diameter 27 and a length 29 which are sized to be introduced through an incision and into the body of a patient, such as into the brain or the epidural space of the spinal column. In one embodiment, diameter 27 of catheter 22 is approximately 1 to 2 mm, and more particularly approximately 1.6 mm, and length 29 is approximately 22 to 30 cm, and more particularly approximately 26 cm. Catheter 22 may be made of a flexible, physiologically inert material such as a medical grade silicone elastomer, polyurethane, polytetrafluoroethylene (PTFE), or the like.

Stylet 23 is positioned within the lumen 21 of catheter 22. Stylet 23 may be fabricated from a semi-rigid material, such as titanium, tungsten or tungsten alloy, to provide some stiffening/structural rigidity to the catheter and aid in passing the catheter 22 into the body of the patient. Stylet 23 includes a proximal end 36 and a distal end 34. The distal end 34 of the stylet 22 includes shoulders 43 that are shaped to push against contact surfaces 43 of end cap 9. In this way, stylet 23 pushes the catheter 22 through the tissue toward its desired location. At least one temporary stimulation electrode 24 is disposed on distal end 34 of stylet 23. A stylet handle 18 is disposed on the proximal end 36 of the stylet 23, which may be used to remove the stylet from the catheter once proper placement of the catheter is achieved.

An extension 10 electrically connects the stylet 23 to a stimulator 28. The stimulator is the source of electrical pulses for electrical test stimulation of the target tissue. In one embodiment, stimulator 28 includes a device external to the body that may be used during a surgical procedure in which catheter 22 is inserted or implanted into the patient. In one embodiment, stylet 23 is electrically conductive and conducts electrical stimulation pulses from the stimulator 28 to the temporary stimulation electrode 24. Temporary stimulation electrode 24 then delivers the electrical test stimulation to the target tissue. Based on the response of the patient to the electrical test stimulation, a physician can determine whether catheter 22 is properly positioned with respect to the target tissue for delivery of a therapeutic substance.

Stylet 23 and its associated temporary stimulation electrode 24 is removably positioned within the lumen 21 of catheter 22. After verification of proper positioning of the catheter with respect to the target tissue, stylet 23 and its associated temporary stimulation electrode 24 are removed from the catheter, leaving catheter 22 in place with respect to the target tissue. Hence, placement of catheter 22 involves a single insertion of catheter 22, rather than insertion of an electrical stimulation lead followed by withdrawal of the lead and insertion of a catheter.

Introduction of a test electrode via a catheter permits quick verification of proper catheter positioning. In particular, patient response to electrical test stimulation can be evaluated almost immediately, in comparison to evaluation of patient response to therapeutic substances, which may require an extended period of time and thereby prolong the placement procedure. Moreover, the invention avoids the need to separately deploy the catheter and an electrode lead. Instead, if the test electrode introduced via the catheter indicates proper positioning, the electrode is withdrawn but the catheter simply remains in place at the target site. The therapeutic substance may then be delivered through the catheter lumen 21 directly to the target tissue.

The catheter 22 may be used to deliver any of a number of therapeutic substances. For example, the catheter 22 may deliver pharmaceutical therapies such as anesthetics or narcotics, biological therapies such as gene therapy agents or growth factors, or other neuropharmacologic agents (i.e., agents that effect neurotransmitter activity or metabolism) suitable for delivery to various parts of the central nervous system. The therapeutic substance delivered may be of a liquid, semi-liquid, or gel-type form which can flow through the lumen 21 of the catheter 22. The therapeutic substance exits one or more ports in or adjacent to the distal end 38 of the catheter 22. For example, the therapeutic substance may be delivered to the target tissue via the opening created by the lumen 21 at the distal end 38 of the catheter 22.

In one embodiment, temporary stimulation electrode 24 is formed by coating at least a portion of the distal end of stylet 24 with a conductive material. The conductive material may include, for example, platinum, platinum-iridium, stainless steel, or some other low impedance, biocompatible conductive material. Although FIG. 1 shows a catheter 22 with one temporary stimulation electrode 24, more than one temporary stimulation electrode 24 may be used, and it shall be understood that the invention is not limited in this respect. For example, the temporary stimulation electrode 24 may include one, two, three or more electrodes. These electrodes 24 may operate as monopolar or bipolar electrodes. For a monopolar embodiment, the temporary stimulation electrode 24 may include one stimulation electrode located at distal end 34 of stylet 23 and a return or reference electrode such as a ground pad attached to the patient's skin. In a bipolar embodiment, the stimulating current flows between at least one source electrode and at least one return electrode, both of which may be located at the distal end 34 of stylet 23. The bipolar electrodes may be provided by a twisted pair, a coaxial bipolar electrode arrangement, or by a conductive coating/ring electrode arrangement. Furthermore, multipolar stimulation arrangements, such as quadripolar, octapolar or other known electrode arrangements are also within the scope of the present invention.

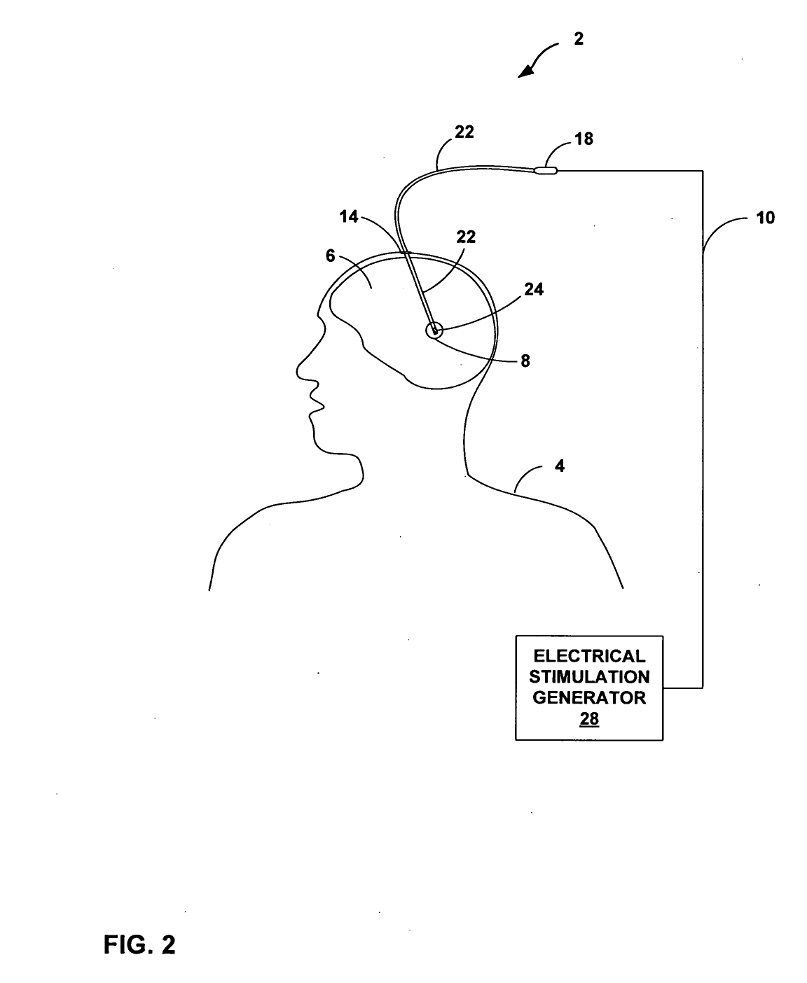

FIG. 2 shows a catheter 22 inserted within a brain 6 of a patient 4. In FIG. 2, the catheter 22 is connected for delivery of electrical test stimulations to verify proper positioning of the catheter with respect to the target tissue 8. Burr hole cap 14 forms a port through the patient's skull for introduction of catheter 22. Catheter 22 includes an interior lumen (not shown) extending through the length of the catheter 22. The lumen may be used for delivery of therapeutic substance to the target tissue 8 within the brain 6 of patient 4. Catheter 22 also includes a stylet (see FIG. 1) removably positioned within the lumen of the catheter. A stylet handle 18 may be used to remove the stylet from the catheter once proper placement of the catheter is achieved. At least one temporary stimulation electrode 24 is positioned at the distal end of stylet 23. The temporary stimulation electrode 24 is connected via the stylet and extension 10 to a stimulator 28. Test stimulations generated by stimulator 28 may be delivered to the target tissue 8 via temporary stimulation electrode 24 to verify whether catheter 22 is properly positioned with respect to the target tissue 8.

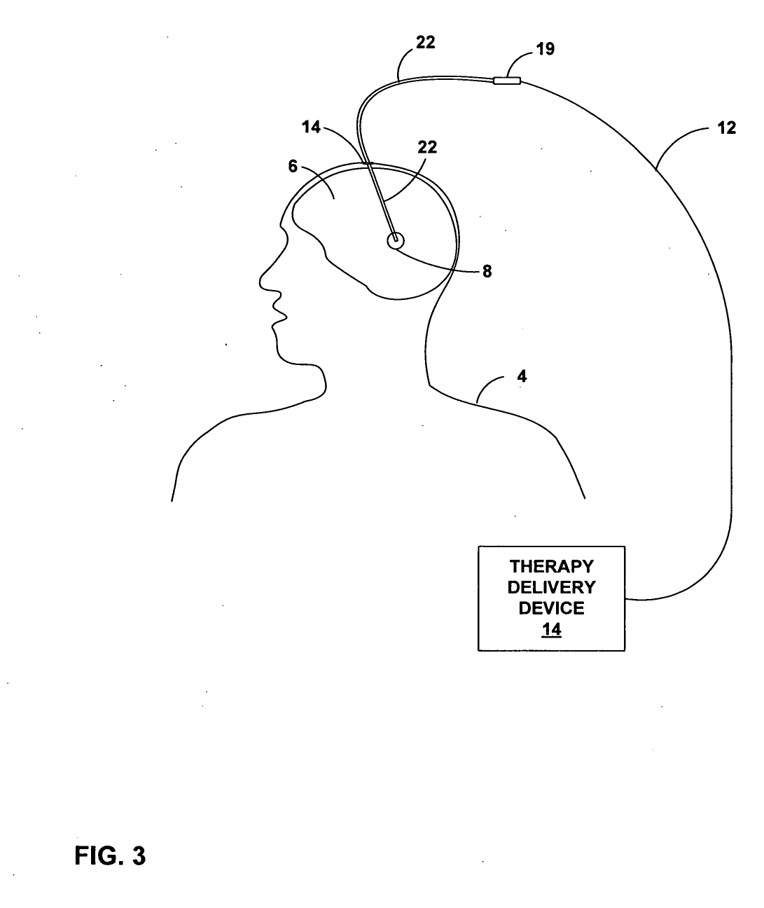

FIG. 3 shows the catheter 22 of FIG. 2 in which target verification has been achieved. The catheter 22 is connected for delivery of a therapeutic substance to target tissue 8 within the brain 6 of patient 4. Burr hole cap 14 forms a port through the patient's skull for introduction of catheter 22. As shown in FIG. 3, the stylet and its associated temporary stimulation electrode have been removed from the lumen of the catheter 22, leaving catheter 22 in place for delivery of one or more therapeutic substances. The catheter includes a lumen (see FIG. 1) extending therethrough for delivery of liquid, semi-liquid or gel-type therapeutic substances to the target tissue. Catheter 22 is connected via connector 19 and fluid delivery conduit 12 to a therapy delivery device 14. Therapy delivery device 14 can include, for example, a hypodermic syringe, drug pump, infusion pump or other suitable device for providing a therapeutic substance to target tissue 8 within the brain 6 of a patient 4. The therapeutic substance may be delivered to brain 5 of patient 3 to treat a variety of neurological symptoms or conditions such as chronic pain, spasticity, dystonia, Parkinson's disease, epilepsy, incontinence, gastro paresis, or to provide pain relief during surgery. The therapeutic substance may also be delivered to reduce pain during a surgical procedure.

In one embodiment, the patient response to the test stimulation can include any one or more of symptom reduction, evoked potentials, subjective sensations, paraesthesia, subjective input from the patient or objective observation by the physician. If the test stimulation causes a patient response consistent with that which would result from proper positioning of catheter 22, it may be determined that catheter 22 is properly positioned with respect to the target tissue 8. If the patient response is not consistent with proper positioning of the catheter 22, catheter 22 may be moved to another position, and another test stimulation may be delivered. This process may be repeated until distal end of catheter 22 is properly positioned with respect to the target tissue. Once catheter 22 is positioned in the desired location, the stylet and its associated temporary stimulation electrode 24 may be withdrawn from the lumen extending through catheter 22, while leaving the catheter 22 in place with respect to the target tissue. At this point, the catheter 22 may deliver one or more therapeutic substances to target tissue 8.

The catheter with temporary stimulation electrode described herein provides more precise means of target verification before injection of a therapeutic substance. The catheter with temporary stimulation electrode therefore may increase the effectiveness of the therapeutic substance and decrease the potential for adverse side effects. Once target verification is achieved via the temporary stimulation electrode, the stylet and the temporary stimulation electrode may be removed without affecting the final placement of the catheter itself. Thus, there is no need to separately introduce a stimulation electrode, remove the stimulation electrode, and reintroduce a catheter once the location of the target tissue has been identified. This increases the accuracy of the catheter's final placement and reduces the number of times the brain or other tissue must be traversed for successful introduction of the catheter. This may result in a decreased risk to the patient and may also reduce patient recovery time.

The catheter with temporary stimulation electrode may be used in any of a number of surgical applications where therapeutic substances are to be delivered. For example, the device may be used for target verification for the delivery of anesthesia or narcotics for pain relief during surgery. The device may also be used for target verification for the delivery of biological agents, such as gene therapy agents or growth factors, into brain tissue during a surgical procedure. As another example, the device may be used for target verification for implantable drug delivery systems. An implantable drug delivery system includes a drug infusion pump implanted in the body of the patient and a catheter implanted in the intrathecal or epidural space of the spinal column, or a catheter implanted within the brain. Typically, the drug delivery system may be refilled periodically by inserting a needle percutaneously into a septum located on the drug infusion pump. It shall be understood that the catheter with temporary stimulation electrode described herein may be used for target verification for virtually any procedure in which a catheter is to be inserted into bodily tissue for the delivery of therapeutic substances, whether the insertion of the catheter into the bodily tissue is temporary (such as during a surgical procedure) or permanent (such as implantation).

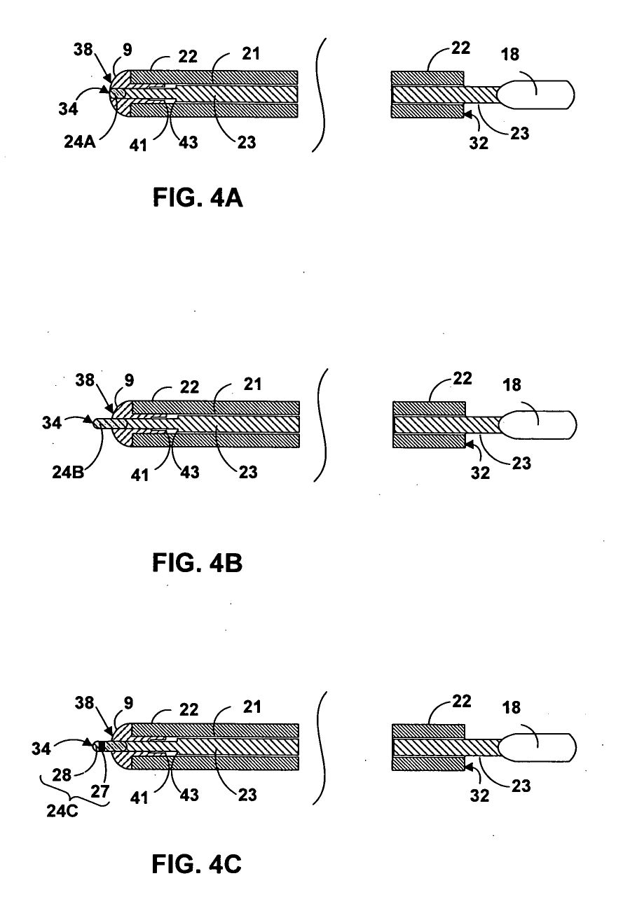

FIGS. 4A-4C are perspective diagrams illustrating catheters 22 having alternate embodiments of temporary stimulation electrodes 24. FIG. 4A illustrates a monopolar electrode 24A located at the distal end 34 of stylet 23. In one embodiment, the monopolar electrode 24A may include a conductive coating disposed on stylet 23. The conductive coating may consist of any suitable coating having a desired conductivity, such as platinum, platinum-iridium, stainless steel, gold or other low impedance, biocompatible conductive material. In another embodiment, the electrode 24A may be formed as a separate piece of conductive material that is then joined to the distal end 38 of stylet 23. The electrode 24A could be joined via standard techniques for joining metals such as welding, use of a conductive adhesive, etc.

In the embodiment of FIG. 4A, the distal end 34 of stylet 23 is substantially flush with the distal end 38 of catheter 22. In this position, monopolar electrode 24A delivers a test stimulation to tissue in contact with the electrode. The return path for the electrical stimulation may be provided via a ground pad (not shown) attached to the patient's skin or via another electrode placed somewhere on or in the patient's body.

FIG. 4B illustrates another example embodiment of a catheter having a temporary stimulation electrode. In this embodiment, the distal end 34 of stylet 22 having a monopolar temporary stimulation electrode 24B extends beyond the distal end 34 of catheter 22. Distal end 34 of stylet 23 may extend anywhere in the range of from 1 millimeters to 5 millimeters beyond the distal end 38 of catheter 22. As with the embodiment of FIG. 4A, the monopolar electrode 24B may include a conductive coating disposed on stylet 23, an electrode material integral with the distal tip of stylet 23, or a separate conductive electrode joined to the distal end 38 of stylet 23. A reference electrode, such as a ground pad attached to the patient's skin or another electrode placed somewhere on or in the patient's body, may serve as the return electrode.

FIG. 4C illustrates another example embodiment of a catheter having a temporary stimulation electrode. In this embodiment, at least one bipolar electrode 24C is disposed at distal end 38 of stylet 23. Bipolar electrode 24C may include a conductively coated electrode 28 as the source electrode and a circumferential ring electrode 27 as the reference electrode. The ring electrode 27 may be electrically connected back to the stimulator via an insulated, electrically conductive wire (not shown) extending back through the catheter lumen 21. Alternatively, a bipolar electrode may also be achieved using two or more circumferential ring electrodes, a coaxial bipolar electrode arrangement, or other bipolar electrode arrangements that are known in the art. As with the monopolar embodiments described with respect to FIGS. 4A and 4B, the distal end 34 of stylet 23 and/or the bipolar electrodes 24C may be substantially flush with or extend some distance beyond the distal end 38 of catheter 22.

It shall be understood that although particular monopolar and bipolar electrode arrangements have been shown and described, many other monopolar or bipolar arrangements known in the art may be substituted for those shown, and that the invention is not limited in this respect. It shall also be understood that alternative methods of stimulation, including multipolar stimulation (e.g., quadripolar, octapolar, etc.) may also be used without departing from the scope of the present invention.

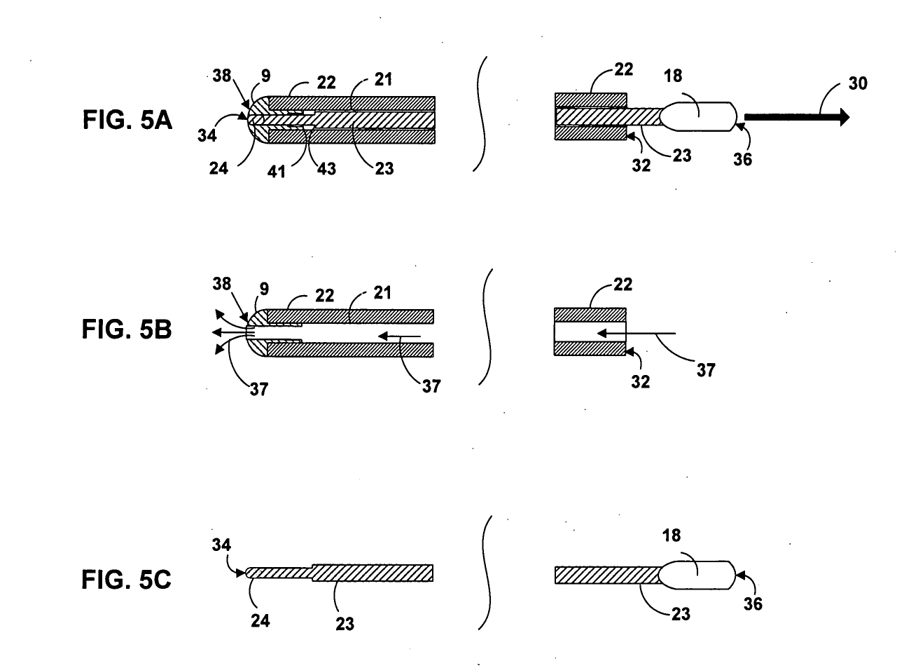

FIG. 5A shows a cross-sectional side view of a catheter 22 having a temporary stimulation electrode 24 removably positioned therein. The catheter includes a lumen 21 extending from the proximal end 32 to the distal end 38 of the catheter 22. A stylet 23 is removably positioned within the lumen 21 of the catheter 22. Temporary stimulation electrode 24 is disposed at the distal end 34 of the stylet 23. A stylet handle 18 is used to remove the stylet 23 along with its temporary stimulation electrode 24 from the catheter 22 by pulling in the direction indicated by arrow 30.

FIG. 5B shows a cross-sectional side view of a catheter 22 which has had its stylet and associated temporary stimulation electrode removed. After removal of the stylet 23 and its associated temporary stimulation electrode 24, the catheter 22 is capable of delivering therapy in the form of liquid, semi-liquid, or gel-type therapeutic substances. The therapy may be introduced at the proximal end 32 of catheter 22, flow through the lumen 21 and delivered from distal end 38 of the catheter 22. Flow of the therapeutic substance through the lumen 21 of catheter 22 is generally indicated by arrows 37.

FIG. 5C shows the removed stylet 23, associated stylet handle 18 and temporary stimulation electrode 24. In one embodiment, stylet 23 may be discarded after use with a single patient. In another embodiment, stylet 23 may be sterilized for future use.

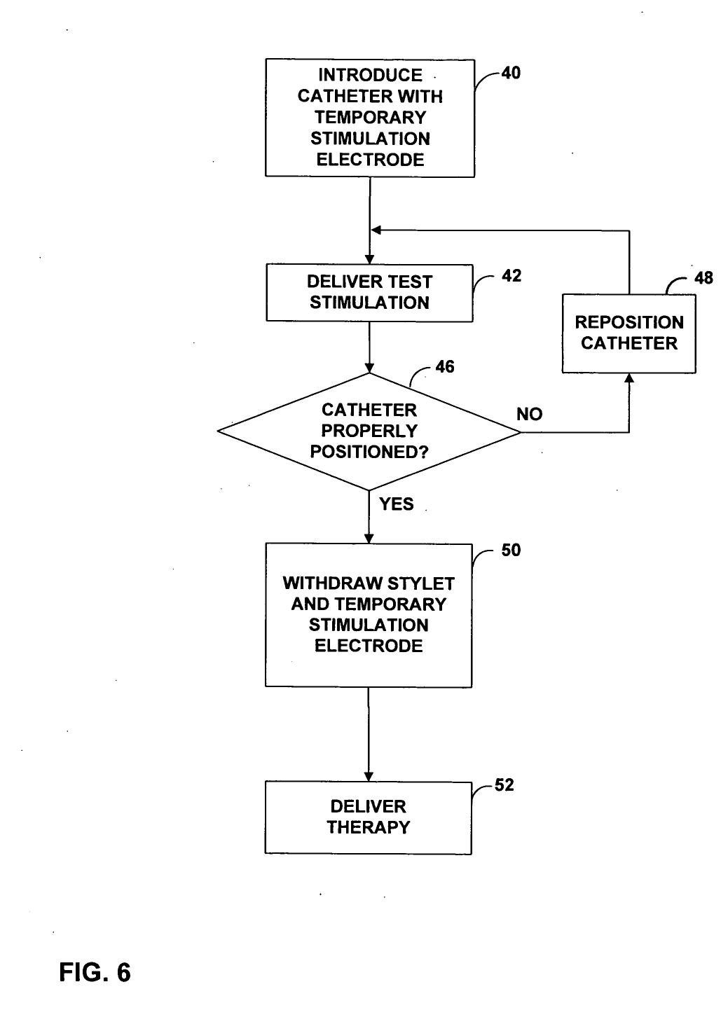

FIG. 6 is a flow diagram illustrating one embodiment of a method of using a catheter with temporary stimulation electrode for target verification and delivery of one or more therapeutic substances. In this embodiment, a catheter having a stylet and its associated temporary stimulation electrode removably positioned therein is introduced into the tissue (40). Once initially positioned, an electrical stimulation generator electrically connected to the stylet delivers a test stimulation via the temporary stimulation electrode (42).

If the patient response to the test stimulation is consistent with accurate target localization (46), i.e., if the test stimulation produced the desired patient response, it may be determined that the catheter is properly positioned with respect to the target tissue. However, if the patient response was not consistent with accurate target localization (46), the catheter may be repositioned (48) and another test stimulation may be delivered to the patient (42). In one embodiment, this process may be repeated until the catheter is properly positioned with respect to the target tissue.

After determining that the distal end of the catheter is positioned correctly, the stylet and its associated temporary stimulation electrode are withdrawn from the lumen of the catheter (50). The catheter remains in place in its proper position with respect to the target tissue. A therapeutic substance may then be delivered to the target tissue via the catheter (52).

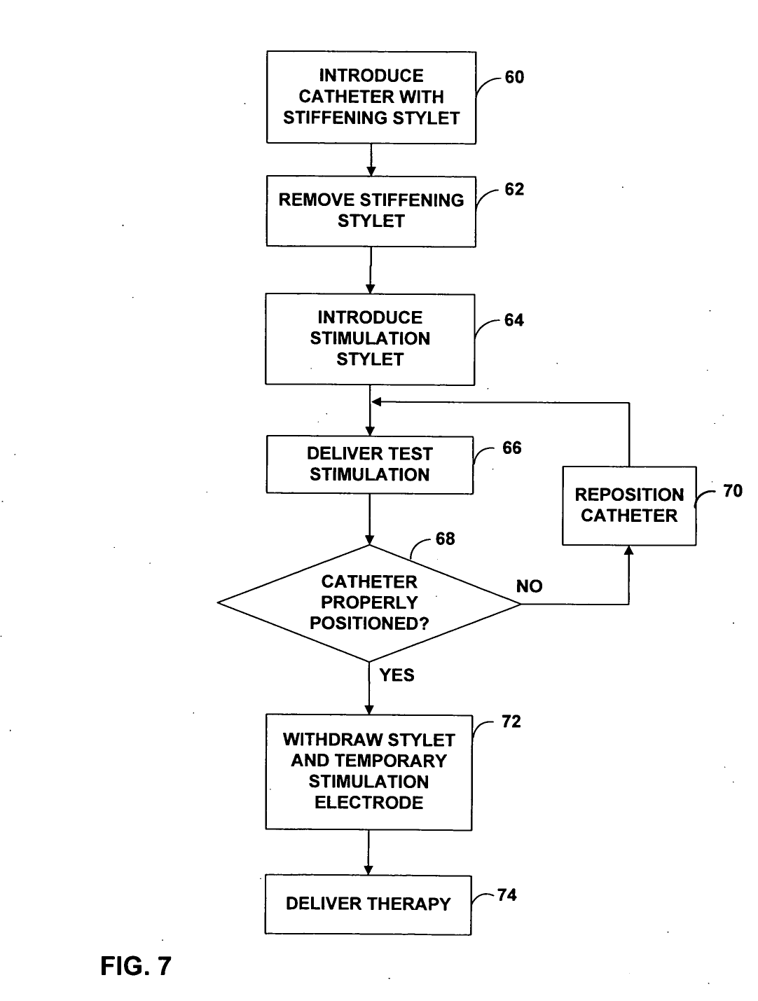

The preceding discussion was directed to embodiments in which the catheter includes a single stylet that performs both the stiffening function (via the material with which the stylet is made, such as tungsten or titanium) and the stimulating function (via the temporary stimulation electrode at the distal end of the stylet). In another embodiment, two separate stylets, a stiffening stylet and a separate stimulation stylet, may be used. In that event, the tiffening stylet and the stimulating stylet could have different mechanical characteristics. For example, the stimulating stylet would not require the same mechanical characteristics, such as stiffness, as would the stiffening stylet. A flow diagram illustrating the two stylet system is shown in FIG. 7.

FIG. 7 is a flow diagram illustrating another embodiment of a method of using a catheter with temporary stimulation electrode for target verification and delivery of one or more therapeutic substances. In this embodiment, a catheter having a stiffening stylet removably positioned therein is introduced into the tissue (60). The stiffening stylet is then removed (62). Next, a separate stimulating stylet having a temporary stimulation electrode at its distal end is introduced into the proximal end of the catheter until the distal end of the stylet reaches the distal end of the catheter (64). As with the embodiments shown in FIGS. 4A-4C, the distal end of the stimulating stylet may be substantially flush with or may extend beyond the distal end of the catheter. An electrical stimulation generator electrically connected to the stimulating stylet delivers a test stimulation via the temporary stimulation electrode (66).

If the patient response to the test stimulation is consistent with accurate target localization (68), i.e., if the test stimulation produced the desired patient response, it may be determined that the catheter is properly positioned with respect to the target tissue. However, if the patient response was not consistent with accurate target localization (68), the catheter may be repositioned (70) and another test stimulation may be delivered to the patient (66). In one embodiment, this process may be repeated until the catheter is properly positioned with respect to the target tissue.

After achieving accurate target localization, the stimulating stylet and its associated temporary stimulation electrode are withdrawn from the catheter (72). The catheter remains in place in its proper position with respect to the target tissue. A therapeutic substance may then be delivered to the target tissue via the catheter (74).

Various embodiments of the invention have been described. However, one skilled in the art will appreciate that various modifications may be made to these embodiments without departing from the scope of the invention. These and other embodiments are within the scope of the following claims.

1. A device comprising:
a catheter having a proximal end and a distal end and a lumen extending between the proximal end and the distal end of the catheter to deliver a therapeutic substance to target tissue;
a stylet removably positioned within the lumen and having a proximal end and a distal end, wherein the distal end of the stylet extends beyond the distal end of the catheter; and
at least one stimulation electrode disposed at the distal end of the stylet to verify proper positioning of the distal end of the catheter with respect to the target tissue.

2. The device of claim 1, further including an electrical stimulation generator coupled to the stimulation electrode to deliver a test stimulation to the target tissue via the stimulation electrode to produce a patient response.

3. The device of claim 1, further including a therapy delivery device coupled to the catheter to deliver a therapeutic agent to the target tissue after removal of the stylet from the lumen.

4. The device of claim 1, wherein the therapeutic substance includes at least one of a pharmaceutical therapy, a biological therapy or a neuropharmaceutical agent.

5. The device of claim 4, wherein the pharmaceutical therapy includes at least one of an anesthetic or a narcotic.

6. The device of claim 4, wherein the biological therapy includes at least one of a gene therapy agent or a growth factor.

7. The device of claim 1, wherein the catheter has a size selected for introduction into a brain or a spinal column of a patient.

8. The device of claim 1, wherein the catheter comprises a flexible material.

9. The device of claim 8, wherein the catheter comprises a medical grade silicon elastomer, a polyurethane, or polytetrafluoroethylene (PTFE).

10. The device of claim 1, wherein the stylet comprises at least one of a tungsten or a titanium material.

11. The device of claim 1, wherein the stimulation electrode includes one of a monopolar electrode, a bipolar electrode, or a multi-polar electrode.

12. The device of claim 1, wherein the distal end of the stylet extends from between 1 millimeters and 5 millimeters beyond the distal end of the catheter.

13. The device of claim 1, wherein the stimulation electrode comprises a conductive layer disposed at the distal end of the stylet.

14. The device of claim 13, wherein the conductive layer comprises a biocompatible material.

15. The device of claim 13, wherein the conductive layer comprises one of platinum, platinum-iridium or stainless steel.

16. The device of claim 1, wherein the stimulation electrode comprises a bipolar electrode including a conductive layer disposed at the distal end of the stylet and a circumferential ring electrode.

17. The device of claim 1, further including an electrical stimulation generator external to the body of the patient.

18. The device of claim 1, further comprising a fluid delivery device to deliver the therapeutic substance to the target tissue via the lumen following removal of the stylet.

19. A method comprising:
introducing a catheter having a temporary stimulation electrode removably positioned therein to target tissue within a body of a patient such that a distal end of the temporary stimulation electrode extends beyond a distal end of the catheter;
delivering a test stimulation via the temporary stimulation electrode to produce a patient response;
determining, based on the patient response, whether the catheter is properly positioned with respect to the target tissue; and
removing the temporary stimulation electrode from the catheter when the catheter is properly positioned.

20. The method of claim 19, further comprising delivering a therapeutic substance to the target tissue via the catheter after removing the temporary stimulation electrode.

21. The method of claim 20, wherein delivering a therapeutic substance includes delivering at least one of a pharmaceutical or a biological therapy.

22. The method of claim 21, wherein delivering a pharmaceutical therapy includes delivering at least one of an anesthetic or narcotic.

23. The method of claim 21, wherein delivering a biological therapy includes delivering a gene therapy agent.

24. The method of claim 19, further comprising:
repositioning the catheter when the patient response is not consistent with proper positioning of the catheter; and
delivering an additional test stimulation via the stimulation electrode.

25. The method of claim 19, wherein the patient response includes one of symptom reduction, evoked potentials, subjective sensations, or paraesthesia.

26. A device comprising:
a catheter having a proximal end and a distal end and a lumen extending between the proximal end and the distal end of the catheter to deliver a therapeutic substance to target tissue;
a stiffening stylet removably positioned within the lumen and having a proximal end and a distal end, wherein the distal end of the stylet extends beyond the distal end of the catheter; and
at least one stimulation electrode disposed at the distal end of the stylet to verify proper positioning of the distal end of the catheter with respect to the target tissue.

27. The device of claim 26, wherein the stylet comprises at least one of a tungsten or a titanium material.

28. The device of claim 26, wherein the stimulation electrode includes one of a monopolar electrode, a bipolar electrode, or a multi-polar electrode.

29. The device of claim 26, wherein the distal end of the stylet is substantially flush with the distal end of the catheter.

30. The device of claim 26, wherein the distal end of the stylet extends beyond the distal end of the catheter.

31. The device of claim 30, wherein the distal end of the stylet extends from between 1 millimeters and 5 millimeters beyond the distal end of the catheter.

32. The device of claim 26, wherein the stimulation electrode comprises a conductive layer disposed at the distal end of the stylet.

33. The device of claim 32, wherein the conductive layer comprises a biocompatible material.

34. The device of claim 32, wherein the conductive layer comprises one of platinum, platinum-iridium or stainless steel.

35. The device of claim 26, wherein the stimulation electrode comprises a bipolar electrode including a conductive layer disposed at the distal end of the stylet and a circumferential ring electrode.

36. A method comprising:
introducing a catheter having a stiffening stylet removably positioned therein to target tissue within a body of a patient;
introducing a stimulation stylet having a temporary stimulation electrode positioned at its distal end into the catheter;
delivering a test stimulation via the temporary stimulation electrode to produce a patient response;
determining, based on the patient response, whether the catheter is properly positioned with respect to the target tissue; and
removing the temporary stimulation electrode from the catheter when the catheter is properly positioned.

37. The method of claim 36, further comprising delivering a therapeutic substance to the target tissue via the catheter after removing the temporary stimulation electrode.

38. The method of claim 37, wherein delivering a therapeutic substance includes delivering at least one of a pharmaceutical or a biological therapy.
